(19) United States
(12) Patent Application Publication
(10) Pub. No.: US 2026/0131124 A1
(43) Pub. Date: May 14, 2026

(54) FILM TUBE FOR AN INTRODUCER SHEATH ASSEMBLY

(71) Applicant: W. L. Gore & Associates, Inc., Newark, DE (US)

(72) Inventors: David J. Arcaro, Flagstaff, AZ (US); Jeffrey B. Duncan, Flagstaff, AZ (US); Thomas R. McDaniel, Flagstaff, AZ (US)

(21) Appl. No.: 19/385,314

(22) Filed: Nov. 11, 2025

Related U.S. Application Data
(60) Provisional application No. 63/719,352, filed on Nov. 12, 2024.

(51) Int. Cl. A61M 39/06; A61M 25/06

(52) U.S. Cl. CPC A61M 39/0613; A61M 25/0662; A61M 2039/0673; A61M 2205/0216; A61M 2205/3331; A61M 2207/00

(57) ABSTRACT

A valve assembly comprising an outer tube, and an inner tube arranged at least partially within the outer tube member, the inner tube is formed of a compliant material and extending along a longitudinal axis between a first end and a second end, and a center portion located between the first and second ends, the center portion defining a center portion outer diameter, a first end ring defining a first end ring outer diameter, the first end ring being defined by a first segment of the inner tube adjacent the first end that has been fixed in a longitudinally compressed state to define the first end ring outer diameter, the first end ring outer diameter being greater than the center portion outer diameter.

### CROSS-REFERENCE TO RELATED APPLICATION

This application claims the benefit of Provisional Application No. 63/719,352, filed Nov. 12, 2024, which is incorporated herein by reference in its entirety for all purposes.

### BACKGROUND

During endoluminal procedures, an insertion site is formed through a patient's skin to gain access to the vasculature of the patient. These insertion sites also provide a path for blood to flow from the patient that needs to be controlled (sealed). Generally, endoluminal procedures utilize introducer sheaths, or introducer sheath assemblies, with some type of hemostasis valve for reducing or preventing blood flow through the introducer sheath, including reducing or preventing blood flow past an endoprosthesis system introduced into the vasculature through the introducer sheath.

### SUMMARY

Various examples addressed in this patent specification relate to introducer sheath valve designs that facilitate one or more of: ease of manufacture, enhanced efficacy, and/or improved reliability. For example, various concepts relate to a valve assembly for an introducer sheath, the valve assembly including an inner tube through which an endoluminal system (e.g., a transcatheter delivery system) may be passed, with the inner tube being able to seal against the endoluminal system. More specifically, various concepts relate to coupling/seal designs for the inner tube that facilitate securing the inner tube within the valve assembly.

For example, some introducer sheath assembly embodiments include a pair of flexible members (e.g., an inner tube and an outer tube arranged coaxially) that are radially separated to create a pressurizable space. In order to effectively seal and couple the tubes relative to one another (e.g., through attachment to an associated housing), the one or more flexible members may include one or more retention features to secure the flexible member(s) and thereby help maintain an operative seal for the pressurizable space. The retention features may be integral with the remainder of the flexible member(s). For example, retention features may be formed by longitudinal compression, eversion or other methods to maintain uniformity (e.g., continuous, homogenous, monolithic, single piece, or other continuous structure) of the flexible member(s). The retention features may be in the form of rings, annuluses, or other circumferentially extending features, for example. The valve description and examples are generally provided in the context of a hemostasis, or hemostatic valve, such as those used in association with an introducer sheath. The valve designs and concepts provided in this patent specification can also be applied to other types of valves, and a variety of applications, and thus the scope of the inventive concepts addressed herein should be read broadly, and to be applicable to a variety of functional valve types.

According to one example (“Example 1”), a valve assembly comprises an outer tube (e.g., an outer tube), and an inner tube (e.g., an inner tube) arranged at least partially within the outer tube, the inner tube is formed is formed of a compliant material and extends along a longitudinal axis between a first end and a second end, and a central portion located between the first and second ends, the center portion defining a center portion outer diameter, a first end ring defining a first end ring outer diameter, the first end ring being defined by a first segment of the inner tube adjacent the first end that has been fixed in a longitudinally compressed state to define the first end ring outer diameter, the first end ring outer diameter being greater than the center portion outer diameter.

According to another example (“Example 2”), further to Example 1, a second end ring is defined by a second segment of the inner tube adjacent the second end that has been longitudinally compressed to define a second end ring outer diameter, the second end ring outer diameter being greater than the center portion outer diameter.

According to another example (“Example 3”), further to Example 2, the valve assembly further includes a first collar and a second collar, the first end ring engaged by the first collar and the second end ring engaged by the second collar.

According to another example (“Example 4”), further to any of the preceding Examples, the outer tube and the inner tube are coupled together to define a pressurizable space.

According to another example (“Example 5”), further to Example 2, the first end ring outer diameter and the second end ring outer diameter are approximately equal.

According to another example (“Example 6”), further to any of the preceding Examples, the first segment of the inner tube has been post-process treated to fix the first segment in the longitudinally compressed state.

According to another example (“Example 7”), further to Example 6, the post-process includes UV crosslinking the first segment.

According to another example (“Example 8”), further to Example 6, the post-process includes chemically treating the first segment.

According to another example (“Example 9”), further to Example 6, the post-process includes heat treating the first segment.

According to another example (“Example 10”), further to any of the preceding Examples, the center portion of the inner tube is formed of a drapeable material, and the first segment has a compression ratio of at least 400%, where the compression ratio is defined as (length before compression/length after compression)×100%.

According to another example (“Example 11”), further to any of the preceding Examples, the outer tube and the inner tube define a pressurizable space, and the outer tube is a flexible material such that upon pressurizing the pressurizable space, the outer tube distends outwardly.

According to another example (“Example 12”), further to any of the preceding Examples, the center portion of the inner tube is operable to reduce in size upon pressurizing the pressurizable space.

According to another example (“Example 13”), further to any of the preceding Examples, the first end and the second end are integrally formed with the center portion.

According to another example (“Example 14”), further to any of the preceding Examples, the inner tube is formed of an expanded polyethylene (ePE).

According to another example (“Example 15”), further to any of the preceding Examples, the inner tube is formed of an Aliphatic Thermoplastic Polyether Polyurethane (ATPU).

According to one example (“Example 16”), an introducer sheath assembly comprises an outer tube defined by a flexible material, an inner tube arranged at least partially within the outer tube, the inner tube extending along a longitudinal axis between a first end and a second end, and a center portion intermediate the first end and the second end, the inner tube is formed of a compliant material and the first end and the second end being fixed in a longitudinally compressed state such that the first end defines a first end ring having a greater diameter than the center portion and the second end defines a second end ring having a greater diameter than the center portion, and a collar coupled to each of the outer tube and the inner tube to define the pressurizable space.

According to another example (“Example 17”), further to Example 16, the first end ring is engaged by the collar.

According to another example (“Example 18”), further to any of Examples 16-17, the first end ring has been heat treated to retain the first end ring in the longitudinally compressed state.

According to another example (“Example 19”), further to any of Examples 16-18, the outer tube includes a fill port, and the fill port is fluidly coupled with the pressurizable space.

According to another example (“Example 20”), further to any of Examples 16-19, the center portion of the inner tube is collapsible to create a seal between the first end and the second end in response to the pressurization of the pressurizable space.

According to another example (“Example 21”), further to any of Examples 16-20, the inner tube is formed of an expanded polyethylene (ePE).

According to another example (“Example 22”), further to any of Examples 16-20, the inner tube is formed of an Aliphatic Thermoplastic Polyether Polyurethane (ATPU).

According to one example (“Example 23”), a method of utilizing an introducer sheath assembly comprises an outer tube, an inner tube extending along a longitudinal axis between a first end and a second end, the inner tube comprising a center portion intermediate the first end and the second end and defining a center portion outer diameter, the inner tube is formed of a compliant material and defines a first end ring adjacent the first end, the first end ring defined by longitudinally compressing and fixing a first segment of the inner tube and the first end ring defining a first end ring outer diameter, a pressurizable space defined between the outer tube and the inner tube, a first collar coupled to each of the outer tube and the inner tube such that the inner tube is engaged by the first collar at the first end ring, and a fill port fluidly coupled to the pressurizable space, the method comprises providing a tool insertable into the inner tube, pressurizing the pressurizable space with a fluid input to the fill port, and reducing the center portion outer diameter in response to the pressurizing such that the center portion sealingly collapses around the tool.

According to another example (“Example 24”), further to Example 23, the outer tube is defined by a flexible material, the method further comprises distending the outer tube in response to the pressurizing.

According to another example (“Example 25”), further to any of Examples 23-24, the inner tube further defines a second end ring adjacent the second end, the second end ring defined by longitudinally compressing and fixing a second segment of the inner tube and the second end ring defining a second end ring outer diameter.

According to another example (“Example 26”), further to Example 25, a second collar is coupled to each of the outer tube and the inner tube such that the inner tube is engaged by the second collar at the second end ring.

According to another example (“Example 27”), further to Example 25, each of the first segment and the second segment are heat treated to fix the first end ring and the second end ring.

According to another example (“Example 28”), further to any of Examples 23-27, the method further comprising depressurizing the pressurizable space by removing fluid from the pressurizable space through the fill port, and distending the inner tube in response to the depressurizing.

According to another example (“Example 29”), further to any of Examples 23-28, the inner tube is formed of an expanded polyethylene (ePE).

According to another example (“Example 30”), further to any of Examples 23-28, the inner tube is formed of an Aliphatic Thermoplastic Polyether Polyurethane (ATPU).

According to one example (“Example 31”), a method of manufacturing a medical device comprises treating an inner tube defining a first end portion, a second end portion, and a central portion such that at least the first end portion is in a reflowable state, wherein the inner tube is drapeable when in a solid state, manipulating the first end portion while in the reflowable state to form a first end ring, arranging the inner tube in an outer tube, and coupling a first collar to the outer tube and the inner tube adjacent the first end ring such that the inner tube and the outer tube define a pressurizable space therebetween.

According to another example (“Example 32”), further to Example 31, further comprising treating the inner tube such that the second end portion is in a reflowable state; and manipulating the second end portion to define a second end ring.

According to another example (“Example 33”), further to any of Examples 31-32, the first end ring is integral with the center portion.

According to another example (“Example 34”), further to any of Examples 31-33, the first end ring defines a first end ring outer diameter, the center portion defines a center portion diameter, and the first end ring outer diameter is greater than the center portion diameter.

According to another example (“Example 35”), further to any of Examples 31-34, the first end ring and the center portion are defined by the same complaint material.

According to another example (“Example 36”) further to any of Examples 31-35, the first end ring is post-process treated to maintain a shape of the first end ring.

According to another example (“Example 37”) further to Example 36, the post-process treatment includes heat treating the first end ring.

According to another example (“Example 38”) further to Example 36, the post-process treatment includes UV crosslinking the first end ring.

According to another example (“Example 39”) further to Example 36, the post-process treatment includes chemically treating the first end ring.

According to another example (“Example 40”), further to any of Examples 31-39, the first end ring is formed by longitudinally compressing the first end portion.

According to another example (“Example 41”), further to any of Examples 31-39, the first end ring is formed by everting a portion of the first end portion.

According to another example (“Example 42”), further to any of Examples 31-41, the inner tube is formed of an expanded polyethylene (ePE).

According to another example (“Example 43”), further to any of Examples 31-41, the inner tube is formed of an Aliphatic Thermoplastic Polyether Polyurethane (ATPU).

The foregoing Examples are just that, and should not be read to limit or otherwise narrow the scope of any of the inventive concepts otherwise provided by the instant disclosure. While multiple examples are disclosed, still other embodiments will become apparent to those skilled in the art from the following detailed description, which shows and describes illustrative examples. Accordingly, the drawings and detailed description are to be regarded as illustrative in nature rather than restrictive in nature.

### BRIEF DESCRIPTION OF THE DRAWINGS

The accompanying drawings are included to provide a further understanding of the disclosure and are incorporated in and constitute a part of this specification, illustrate embodiments, and together with the description serve to explain the principles of the disclosure.

FIG. 1 is a perspective view of an introducer sheath in accordance with an embodiment of the present disclosure;

FIG. 2 is a perspective view of the introducer sheath of FIG. 1;

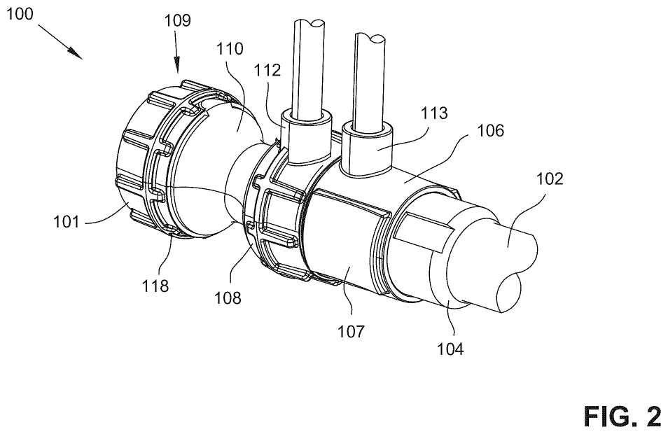

FIG. 3 is an exploded view of the introducer sheath of FIG. 1;

FIG. 4 is a section view of the introducer sheath of FIG. 1;

FIG. 5 is an end on view of the introducer sheath of FIG. 1;

FIG. 6 is a side view of a tube extruded onto a mandrel in accordance with an embodiment of the present disclosure;

FIG. 7 is a side view of the tube of FIG. 6 with each end longitudinally compressed in a first state;

FIG. 8 is a side view of the tube of FIG. 6 with each end longitudinally compressed in a second state;

FIG. 9 is a side view of the tube of FIG. 6 with each end longitudinally compressed to create end rings in accordance with an embodiment of the present disclosure; and

FIG. 10 is a side view of the tube of FIG. 6 with each end everted to create end rings in accordance with an embodiment of the present disclosure.

### DETAILED DESCRIPTION

### Definitions and Terminology

This disclosure is not meant to be read in a restrictive manner. For example, the terminology used in the application should be read broadly in the context of the meaning those in the field would attribute such terminology.

With respect to terminology of inexactitude, the terms “about” and “approximately” may be used, interchangeably, to refer to a measurement that includes the stated measurement and that also includes any measurements that are reasonably close to the stated measurement. Measurements that are reasonably close to the stated measurement deviate from the stated measurement by a reasonably small amount as understood and readily ascertained by individuals having ordinary skill in the relevant arts. Such deviations may be attributable to measurement error, differences in measurement and/or manufacturing equipment calibration, human error in reading and/or setting measurements, minor adjustments made to optimize performance and/or structural parameters in view of differences in measurements associated with other components, particular implementation scenarios, imprecise adjustment and/or manipulation of objects by a person or machine, and/or the like, for example. In the event it is determined that individuals having ordinary skill in the relevant arts would not readily ascertain values for such reasonably small differences, the terms “about” and “approximately” can be understood to mean plus or minus 10% of the stated value.

The term “drape” or “drapeable” can be understood to mean the way a fabric or a material falls when hung in different positions such as the ability of the fabric or material to conform to the shape of another object when laid upon it. Drape is associated with flexibility and suppleness of a fabric or a material.

### Description of Various Embodiments

Persons skilled in the art will readily appreciate that various aspects of the present disclosure can be realized by any number of methods and apparatuses configured to perform the intended functions. It should also be noted that the accompanying drawing figures referred to herein are not necessarily drawn to scale, but may be exaggerated to illustrate various aspects of the present disclosure, and in that regard, the drawing figures should not be construed as limiting.

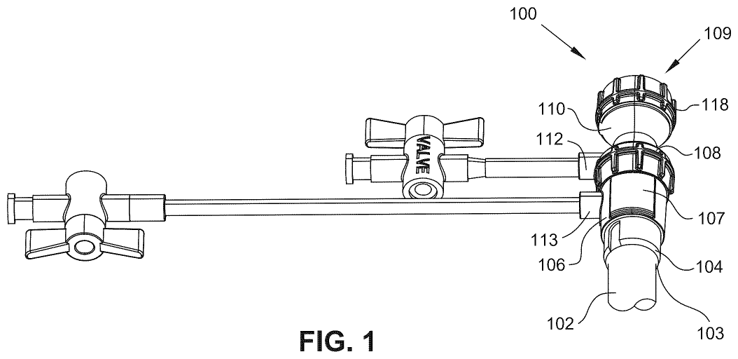

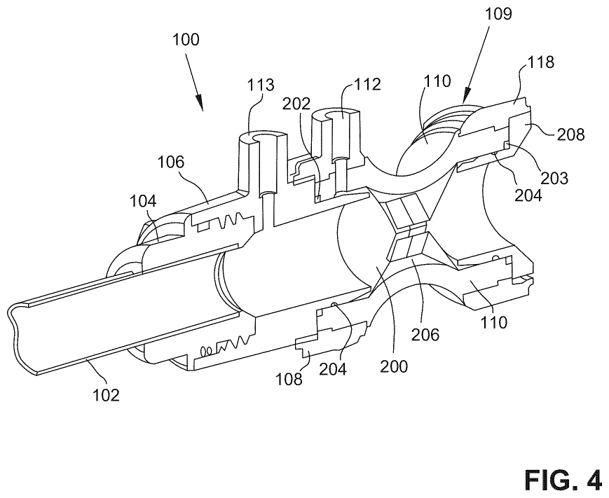

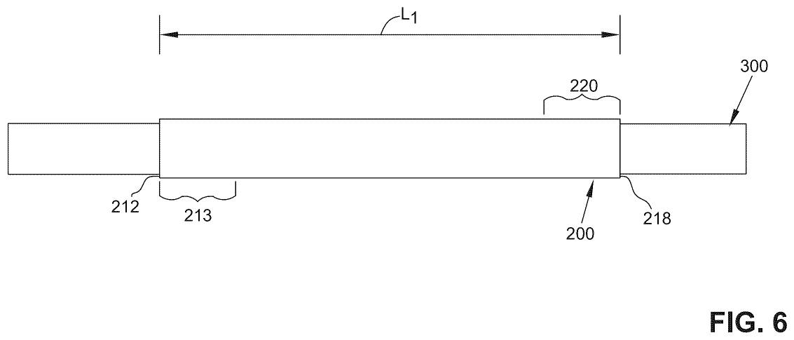

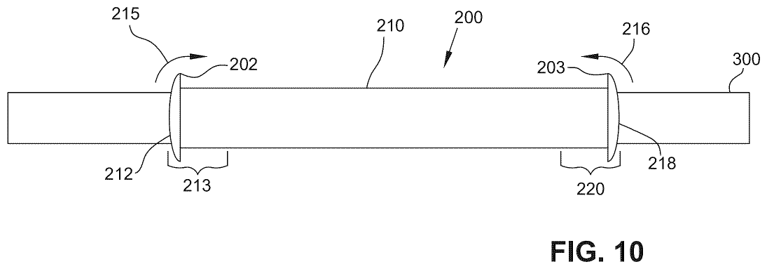

The introducer sheath assembly shown in FIG. 1 is provided as an example of the various features of the introducer sheath assembly and, although the combination of those illustrated features is clearly within the scope of invention, that example and its illustration is not meant to suggest the inventive concepts provided herein are limited from fewer features, additional features, or alternative features to one or more of those features shown in FIG. 1. For example, in various embodiments, the inner tube assembly of the introducer sheath assembly shown in FIG. 4 may include the inner tube with longitudinally compressed rings described with reference to FIGS. 6-9. As another example, the inner tube assembly of the introducer sheath assembly shown in FIG. 4 may include the inner tube with everted rings described with reference to FIG. 10.

Referring to FIG. 1, in accordance with an embodiment, a valve assembly is an introducer sheath valve assembly 100 which is a medical device that includes an introducer sheath 102. The introducer sheath 102 may be formed any material with suitable biocompatible and mechanical properties, for example, fluorinated ethylene propylene (FEP), expanded high density polyethylene (eHDP), or any other material with suitable biocompatible and mechanical properties. The introducer sheath 102 may be of any size including but not limited to from about 12 Fr to 26 Fr or any size therebetween. The introducer sheath valve assembly 100 includes a threaded adapter 104 on a first end 103 of the introducer sheath 102. The threaded adapter 104 may be formed of any biocompatible plastic or any biocompatible metal with suitable biocompatible and mechanical properties. The threaded adapter 104 may be coupled to the introducer sheath 102 by a variety of means which may include, but is not limited to, adhesives such as polyurethan adhesives, quick setting cyanoacrylate adhesives, or ultraviolet cured adhesives, bonding such as thermal bonding, fusing, mechanical adapters, ultrasonic welding, by an interference fit, an insert molding, and so forth.

Still referring to FIG. 1, a front fitting 106 (e.g., a housing) may be coupled to a first end 105 of the threaded adapter 104. The front fitting 106 may be designed to include a profile to facilitate a user gripping the introducer sheath valve assembly 100 securely. The front fitting 106 may include one or more protrusions 107 to enhance traction for a user on the introducer sheath valve assembly 100. In some embodiments, the front fitting 106 may be formed with any biocompatible metal or plastic with suitable biocompatible and mechanical properties, and the protrusions 107 may be made of a similar material as the front fitting 106 or may be made of a material with a high coefficient of friction, a material more compliant than the front fitting 106, or made with grating, a roughening, a raised company logo or design, or striations in the surface in conjunction with the material listed above to further aid in the gripping of the device. These features on the surface of the front fitting 106 may also be used to aid in gripping without the use of protrusions 107 and may be applied directly to a lateral surface (e.g., a gripping or contact surface) of the front fitting 106. In some embodiments, a flush port 113 may extend from the front fitting 106 (e.g., extends outwardly from a lateral surface of the front fitting 106).

Still referring to FIG. 1, an outer tubular assembly 109 includes a first collar 108 coupled to the front fitting 106, a second collar 118 positioned at a first end 101 of the introducer sheath valve assembly 100, and an outer tube 110 coupled between the first collar 108 and the second collar 118. In some embodiments, the first collar 108 includes a fill port 112, and the fill port 112 is fluidly coupled with an interior of the outer tubular assembly 109. In some embodiments, each of the first collar 108, the second collar 118, and the fill port 112 may be formed of any biocompatible metal or plastic with suitable biocompatible and mechanical properties. In some embodiments, the fill port 112 may be located at any point along either of the first collar 108 or the second collar 118. Referring to FIG. 4, a rear fitting 208 fits within the outer tube 110 and the second collar 118. In some embodiments, the second collar 118 and the rear fitting 208 are integral or otherwise coupled together (e.g., by an adhesive, threads, or other coupling method) to create the second collar 118.

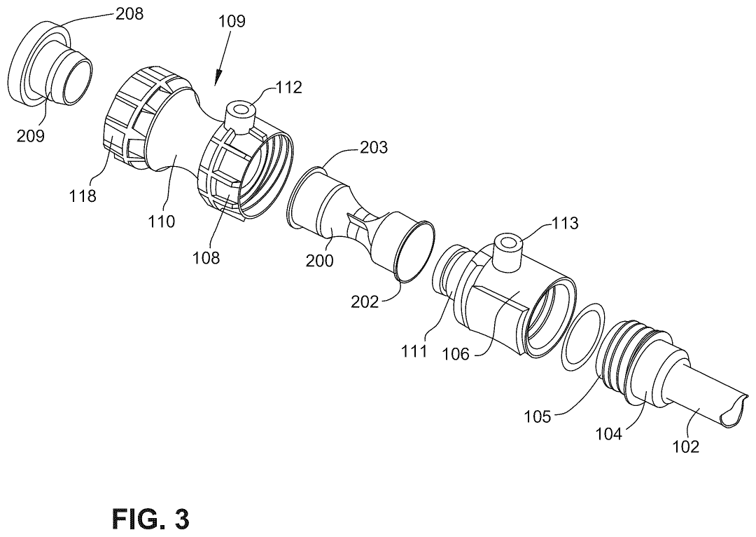

Referring now to FIGS. 3-4, the introducer sheath valve assembly 100 includes an inner tube 200 (e.g., a film tube) arranged inside the outer tubular assembly 109 and a pressurizable space 206 is defined between the outer tubular assembly 109 and the inner tube 200. The inner tube 200 extends along a longitudinal axis and includes a pair of end rings including a first retention feature in the form of a first end ring 202 and a second retention feature in the form of a second end ring 203. Referring to FIG. 4, the inner tube 200 is affixed between the outer tubular assembly 109 and the front fitting 106 at the first end ring 202 and between the outer tubular assembly 109 and the rear fitting 208 at the second end ring 203.

Referring to FIGS. 3-4, the outer tubular assembly 109 includes a pair of sealing lips 204 which may interface with each of the inner tube 200, the front fitting 106, and the rear fitting 208.

In some embodiments, the outer tube 110 may be constructed of any flexible material having desirable mechanical and biocompatible properties, including but not limited to any elastomer, latex, or polycarbonate with desirable mechanical and biocompatible properties. In one embodiment, the outer tube 110 comprises silicone and has an hourglass shape when not pressurized. When the pressurizable space 206 is pressurized to a sufficient pressure such that the inner tube 200 collapses, the outer tube 110 may subsequently distend such that the hourglass shape of the outer tube 110 alters and provides indication of sufficient pressure in the pressurizable space 206 (e.g., when the pressurizable space 206 is pressurized with at least one substance to a sufficient pressure, the inner tube 200 collapses around a profile of any object positioned or inserted therethrough, for example, a tool or another endoluminal device, which limits back bleeding). This feature of the outer tube 110 facilitates the user of a device to easily and quickly identify the optimal pressure for the device. In embodiments, pressurizable space 206 may be depressurized by removing fluid from pressurizable space (i.e., removing fluid through fill port 112) and inner tube 200 may distend back to an original state and outer tube 110 may collapse back to an original state in response to the depressurization.

FIGS. 3 and 4 illustrate the end rings 202, 203 on either end of the inner tube 200. That is, the end rings 202, 203 are separated by a center portion 210 such that center portion 210 is intermediate the end rings 202, 203. In embodiments, center portion 210 is integrally formed with end rings 202, 203. The end rings 202, 203 serve as retention members to provide a stable point of attachment. For example, the end rings 202, 203 may be relatively stiffer than adjacent portions of the inner tube 200, or less compliant, and may be used to aid in attachment and/or sealing of the inner tube 200 to the front fitting 106 and to the rear fitting 208. The end rings 202, 203 may be made of any material with desirable properties (e.g., biocompatible and mechanical properties) and will be described in greater detail below. The inner tube 200 with attached end rings 202, 203 may be inserted through the outer tube 110 and attached to the protruding end 111 of the front fitting 106. The first collar 108 with the fill port 112 may then be attached to the front fitting 106 with an adhesive, mechanical fit (e.g., compression fit), and/or other attachment mechanism. End ring 203 may similarly be attached to the protrusion 209 on the rear fitting 208. The second collar 118 may be snapped, or snap-fit, onto the rear fitting 208. That is, the first collar 108 may be used to mechanically constrain or engage the first end ring 202 and the second collar 118 may be used to mechanically constrain or engage the second end ring 203.

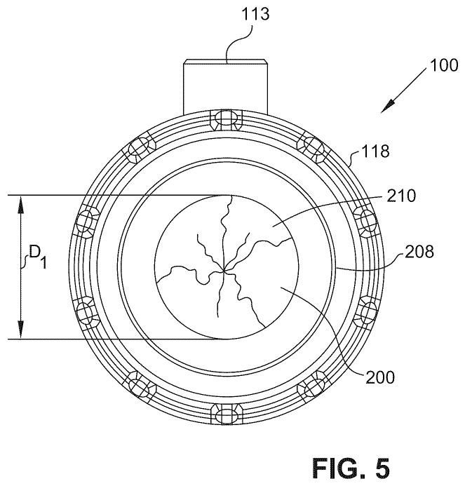

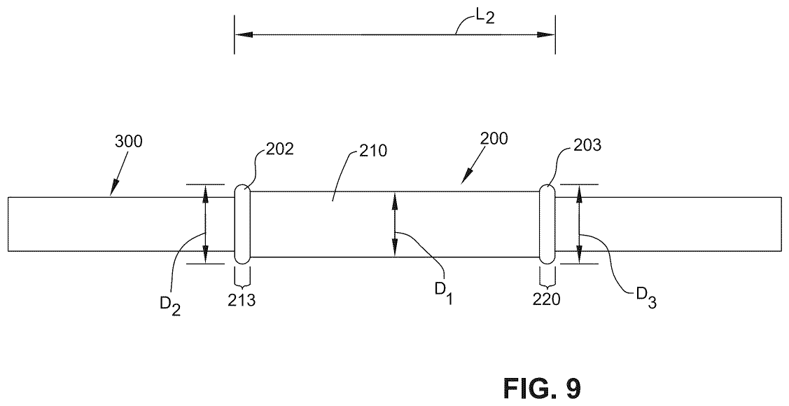

FIG. 5 illustrates an end on view of the device showing a collapsed inner tube 200. In terms of operation, the second collar 118 with the fill port 112 has a feature that allows the pressurizable space 206 to be filled to a sufficient pressure to cause the inner tube 200 to collapse (e.g., at the center portion 210) around a profile of any object positioned or inserted therethrough, for example, a tool or another endoluminal device (e.g., the fill port 112 is fluidly coupled with pressurizable space 206 to allow fluid to move in and out of the pressurizable space 206 (e.g., by a user input or user manipulation)). The pressurizable space 206 may be filled with any suitable material or materials. For example, the pressurizable space 206 may be filled with one or more of the following substances: air, silicone, water, saline solution, low volatility biocompatible liquids, glycerin, propylene glycol, polyethylene glycol, compressible foam, elastomeric spheres, and crosslinked silicone gels. As seen in FIG. 5, the center portion 210 of the inner tube 200 (also FIG. 9) that defines a center portion outer diameter D1 (e.g., an outer diameter taken from the outer surface of center portion 210; also FIG. 9) that varies with the pressure of the pressurizable space 206 (e.g., the lumen has an effective lumen that varies with the pressure of the pressurizable space 206). That is, as the pressure within the pressurizable space 206 increases, the center portion outer diameter D1 or effective lumen of inner tube 200 is operable to decrease such that inner tube 200 constricts or transitions toward a collapsed configuration at the center portion 210. As the inner tube 200 constricts or collapses, or otherwise distends, the inner tube 200 may contact and conform, or drape, around a profile of any object positioned or inserted therethrough, for example, a tool or another endoluminal device, to prevent back bleeding through the inner tube 200.

Inner tube 200 may be constructed of any very thin, strong, drapeable material such as expanded polyethylene (ePE), expanded polytetrafluoroethylene (ePTFE), FEP, fabrics, silk, or Kevlar® brand fiber, and combinations thereof. These materials may be used as a single layer construct or a multi-layer construct, and may be formed as composite materials. For example, the inner tube 200 may be formed of a thin, porous polymeric substrate including multiple layers of material, which may be filled or imbibed with a secondary polymer. The secondary, filling or imbibing polymer may be the same as or similar to that of the construct or may be a different polymer.

Referring now to FIGS. 6-9, the inner tube 200 may be formed on a mandrel 300. The mandrel 300 may be formed of any suitable metal, including coated materials, or other suitable material. In various examples, the inner tube 200 is extruded, wrapped, or otherwise formed onto the mandrel 300 and subsequently removed. As previously referenced, the inner tube 200 may be a film tube formed of a thin, strong, drapeable material (e.g., a compliant material), and in the case of mandrel formation that material will generally be capable of being formed onto the mandrel 300 (e.g., in a flowable state or a solid state). In embodiments, only a portion (e.g., center portion 210) of inner tube 200 is formed of a compliant material and the other portions (e.g., the first end and second) or formed of a less compliant or a non-compliant material. In various examples, the material is malleable during formation of the inner tube 200 and is capable of being formed around, and on the mandrel 300. Following formation, or both following and before formation, the inner tube 200 may be generally compliant in a solid state. The compliant material may be a material capable of elastically deforming and collapsing around or draping around—and creating a seal around—a tool inserted through the introducer sheath valve assembly 100 under an increased pressure within the pressurizable space 206. The compliant material may be an inelastic material capable of deforming and collapsing around or draping around—and creating a seal around—a tool inserted through the introducer sheath valve assembly 100 under an increased pressure within the pressurizable space 206. Regardless of the specific formation method, the inner tube 200 may be formed of a variety of compliant materials, including any of those previously described, which have suitable biocompatible and mechanical properties for the desired application (e.g., expanded polyethylene (ePE)).

Further, the inner tube 200 may be formed of a material capable of being in a solid state and further capable of being reflowable after being molded and cured (e.g., molded into a desired form and cured, and subsequently acted upon to create a reflowed state to allow manipulation of the desired form). In some embodiments, the inner tube 200 is formed of an expanded polyethylene (ePE) which has suitable biocompatible and mechanical properties. In some embodiments, the inner tube 200 is formed of an Aliphatic Thermoplastic Polyether Polyurethane (ATPU) which may have suitable biocompatible and mechanical properties. In some embodiments, another material may be used that has similar biocompatible and mechanical properties as ePE and ATPU. In some embodiments, the inner tube 200 is formed of expanded polytetrafluoroethylene (ePTFE) which may have suitable biocompatible and mechanical properties. The inner tube 200 may be a compliant and deformable within the introducer sheath valve assembly 100 when pressurized to allow for proper sealing (to itself and to a device inserted through the valve) to prevent back bleeding while also having appropriate characteristics so that it may be formed and reflowed at a desirable temperature (e.g., a relatively lower melting point temperature, such as less than 200° C.) to prevent or reduce degradation of the microstructure of the material during the manufacturing process.

Referring to FIG. 6, the inner tube 200 may be formed, or extruded (e.g., by an extruder and/or in an extruding process) on the mandrel 300 between a first end 212 and a second end 218. In some embodiments, the length between the first end 212 and the second end 218 is a first predetermined length L1. In some embodiments, the inner tube 200 is extruded onto the mandrel 300 to a length greater than L1, and the inner tube 200 is cut down to the predetermined length L1. In some embodiments, the mandrel 300 has a non-stick coating to help assist in the removal of the inner tube 200 from the mandrel 300. In some embodiments, the inner tube 200 is formed of a material (e.g., expanded polyethylene (ePE), aliphatic thermoplastic polyether polyurethane (ATPU), expanded polytetrafluoroethylene (ePTFE) which includes end groups which have lubricous qualities and inner tube 200 may more easily be removed from the mandrel 300 as a result of the reduced coefficient of friction between the inner tube 200 and the mandrel 300. Additionally or alternatively, the inner tube 200 may be treated (coated, surface modified, imbibed, or other treatment) to enhance lubricity. Examples of lubricious materials that may be applied to the inner tube 200 include hydrophilic coatings, for example.

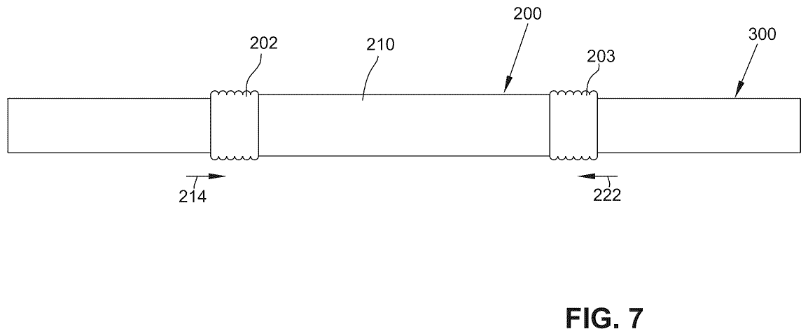

Referring to FIGS. 7-9, in some embodiments, the end rings 202, 203 may be formed after the inner tube 200 is initially formed (e.g., including curing steps, or the like). In other examples, the end rings 202, 203 may be formed at the same time the inner tube 200 is formed. Regardless, in some examples, a first segment 213 (e.g., a first portion) of the inner tube 200 adjacent the first end 212 and a second segment 220 (e.g., a second portion) of the inner tube 200 adjacent the second end 218 are manipulated, or reformed, (e.g., reflowed) to achieve desired properties for the end rings 202, 203. In some embodiments, each of the first segment 213 and the second segment 220 may be reflowed by reheating one or both of the first segment 213 and/or the second segment 220 to a suitable temperature to allow the material to be formable and malleable. In some embodiments, one or both of the first segment 213 and the second segment 220 may be reflowed by a solvent or other chemical treatment that increases the malleability, at least temporarily, of the first segment 213 and the second segment 220.

In some embodiments, the inner tube 200 is formed on the mandrel 300 to the predetermined length L1, and the end ring 202 may be formed by bulking the first segment 213 of the inner tube 200 adjacent the first end 212, and the end ring 203 may be formed by bulking the second segment 220 of the inner tube 200 adjacent the second end 218. In some embodiments, a mechanical tool (e.g., a pusher) may be utilized to bunch up, or longitudinally compress, the first segment 213 and the second segment 220 to create the first end ring 202 and the second end ring 203.

Referring now to FIG. 7, the end rings 202, 203 may be formed by longitudinally compressing the first segment 213 adjacent the first end 212 in the first direction 214 to bulk up the existing material of the first segment 213 into the end ring 202. The end ring 203 may be formed by compressing the second segment 220 adjacent the second end 218 in a second direction 222 to bulk up the existing material of the second segment 220. That is, the first end ring 202 may be defined by the first segment 213 and the second end ring 203 may be defined by the second segment 220. In embodiments, end rings 202, 203 may be positioned adjacent first end 212 and second end 218. In embodiments, end rings 202, 203 may be offset from first end 212 and second end 218, respectively.

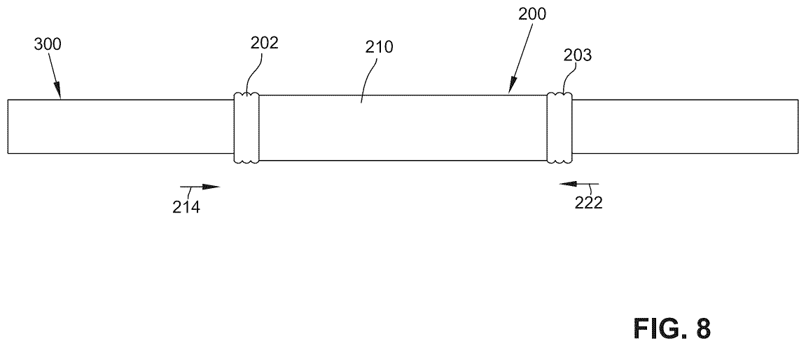

FIGS. 7-9 illustrate the compression of the first segment 213 and the second segment 220 to create the first end ring 202 and the second end ring 203 such that the inner tube 200 has a final length L2. That is, FIGS. 7-8 illustrate the inner tube 200 with each of the first segment 213 and the second segment 220 at different stages of compression, and FIG. 9 illustrates the inner tube 200 with each of the first segment 213 and the second segment 220 compressed into the end rings 202, 203, respectively. That is, first segment 213 and second segment 220 may be longitudinally compressed to create end rings 202, 203 and fixed in the longitudinally compressed state (e.g., FIG. 9). In a first state (e.g., FIG. 6), first segment 213 and second segment 220 may be uncompressed, in a second state (e.g., FIG. 7), first segment 213 and second segment 220 may be partially compressed, and in a third state (e.g., FIG. 9), first segment 213 and second segment 220 may be compressed into the end rings 202, 203, respectively. Illustratively, FIG. 9 shows a final compressed state of the inner tube 200, and the center portion 210 defines the center portion outer diameter D1, the first end ring 202 defines a first end ring outer diameter D2, and the second end ring 203 defines a second end ring outer diameter D3. In some embodiments, the second end ring outer diameter D2 is approximately equal to, or equal to the third end ring outer diameter D3. In some embodiments, each of the second end ring outer diameter D2 and the third end ring outer diameter D3 are greater than the center portion outer diameter D1.

In some embodiments, the first segment 213 and the second segment 220 have a compression ratio defined as the ‘Length Before Compression’ (FIG. 6) divided by the ‘Length After Compression’ (FIG. 9) (i.e., ‘Length Before Compression/Length After Compression’). In some embodiments, the compression ratio is approximately 4:1. In some embodiments, the compression ratio is approximately 10:1. In some embodiments, the compression ratio is approximately 4:1 to 10:1. In some embodiments, the compression ratio is defined as [“(Length Before Compression) divided by (Length After Compression)”×100]% (i.e., [(Length Before Compression/Length After Compression)×100]%). In some embodiments, the compression ratio is approximately 400%. In some embodiments, the compression ratio is approximately 1,000%. In some embodiments, the compression ratio is between 400%-1000%.

In some embodiments, the tube has a pre-compression cross-section and a post-compression cross-section with a resultant aspect ratio of approximately 1:1. For example, in some embodiments, the tube has a pre-compression cross-section that is approximately circular and has a post-compression cross-section that is approximately circular to maintain an approximately 1:1 aspect ratio. In some embodiments, the tube has a pre-compression cross-section that is approximately square and has a post-compression cross-section that is approximately square to maintain an approximately 1:1 aspect ratio.

Referring still to FIGS. 6-9, in some embodiments, at least a portion of the inner tube 200 is capable of being reheated and reflowed. In some embodiments, only a portion of the inner tube 200 is subjected to an increased temperature (e.g., greater than the melting point of the inner tube 200 material) to affect the reheating and reflowing of the portion of the inner tube. In some embodiments, the material chosen (e.g., ePE, ATPU, ePTFE) has a lower melting point relative to previously utilized materials. Lower melting points (e.g., less than 200° C.) are easier to reach and sustain in a manufacturing process (e.g., less energy is required to reach and sustain lower temperatures).

As indicated in FIG. 10, the end rings 202, 203 may also be formed on inner tube 200 by everting the first end 212 and the second end 218 to roll-up or otherwise bunch the first segment 213 and second segment 220, respectively. That is, the first segment 213 adjacent the first end 212 is rolled up (e.g., rotated) in direction 215 to create the end ring 202 and the second segment 220 adjacent the second end 218 is rolled up (e.g., rotated) in direction 216 to create the end ring 203. The two techniques, bunching or longitudinally compressing and everting (or inverting) may be combined as desired to form the ring(s).

In some embodiments, the first end ring 202 and the second end ring 203 are treated after being formed (e.g., compressed and/or everted). In some embodiments, the first end ring 202 and the second end ring 203 are heat treated after compression (e.g., FIGS. 7-8) and/or after eversion (e.g., FIG. 10) such that the first end ring 202 and the second end ring 203 retain their final form (e.g., FIG. 9).

In some embodiments, the first end ring 202 and the second end ring 203 are treated after being formed (e.g., compressed and/or everted) with a localized process that may fuse the end rings 202, 203 while not affecting the center portion (e.g., the center portion 210) of the inner tube 200. In some embodiments, the first end ring 202 and the second end ring 203 are treated after being formed with an ultrasonic welding process to fuse the end rings 202, 203 together. In some embodiments, the first end ring 202 and the second end ring 203 are treated after being formed with a targeted laser process to fuse the end rings 202, 203 together.

In some embodiments, the first end ring 202 and the second end ring 203 are treated after being formed with an induction heating process to fuse the end rings 202, 203 together. Targeted, or localized, treatment of the end rings 202, 203 prevents or minimizes degradation, overheating, or otherwise altering the properties of the material of inner tube 200 in undesired locations.

In some embodiments, the first segment 213 and the second segment 220 may be pre-treated to assist in the creation of the end rings 202, 203.

Such pre-treatment includes laminating, coating, or imbibing the first segment 213 and/or the second segment 220 with an adhesive, for example. When the first segment 213 and the second segment 220 are formed (e.g., compressed, rolled up, and/or everted), the shape, or form of the end rings 202, 203 may be set by the activating the adhesive (e.g., curing, reflowing, cross-linking) on the first segment 213 and/or the second segment 220.

In some embodiments, the first segment 213 and the second segment 220 may be post-treated to assist in creation of the end rings 202, 203. Such post-treatment includes laminating, coating, or imbibing the first segment 213 and/or the second segment 220 with an adhesive, for example. Further post-treatment may include UV crosslinking, chemical treatment, heat treatment, or another post-process treatment. When the first segment 213 and the second segment 220 are formed (e.g., compressed, rolled up, and/or everted), the shape, or form of the end rings 202, 203 may be set or fixed by the activating of the adhesive (e.g., curing, reflowing, cross-linking) on the first segment 213 and/or the second segment 220. That is, the first segment 213 and the second segment 220 may be formed into end rings 202, 203 and subsequently set or fixed in the longitudinally compressed state by a post-treatment.

The invention of this application has been described above both generically and with regard to specific embodiments. It will be apparent to those skilled in the art that various modifications and variations can be made in the embodiments without departing from the scope of the disclosure. Thus, it is intended that the embodiments cover the modifications and variations of this invention provided they come within the scope of the appended claims and their equivalents.

What is claimed is:

1. A valve assembly, comprising:
an outer tube; and
an inner tube arranged at least partially within the outer tube, the inner tube is formed of a compliant material and extends along a longitudinal axis between a first end and a second end, and a center portion located between the first and second ends, the center portion defining a center portion outer diameter, a first end ring defining a first end ring outer diameter, the first end ring being defined by a first segment of the inner tube adjacent the first end that has been fixed in a longitudinally compressed state to define the first end ring outer diameter, the first end ring outer diameter being greater than the center portion outer diameter.

2. The valve assembly of claim 1, wherein a second end ring is defined by a second segment of the inner tube adjacent the second end that has been longitudinally compressed to define a second end ring outer diameter, the second end ring outer diameter being greater than the center portion outer diameter.

3. The valve assembly of claim 2, wherein the outer tube and the inner tube are coupled together to define a pressurizable space and the valve assembly further includes a first collar and a second collar, the first end ring engaged by the first collar and the second end ring engaged by the second collar.

4. The valve assembly of claim 1, wherein the first segment of the inner tube has been post-process treated to fix the first segment in the longitudinally compressed state.

5. The valve assembly of claim 4, wherein the post-process includes one of UV crosslinking the first segment, chemically treating the first segment, or heat treating the first segment.

6. The valve assembly of claim 1, wherein the center portion of the inner tube is formed of a drapeable material, and the first segment has a compression ratio of at least 400%, where the compression ratio is defined as (length before compression/length after compression)×100%.

7. The valve assembly of claim 1, wherein the outer tube and the inner tube define a pressurizable space, and the outer tube is a flexible material such that upon pressurizing the pressurizable space, the outer tube distends outwardly and the center portion outer diameter of the inner tube is operable to reduce in size upon pressurizing the pressurizable space.

8. The valve assembly of claim 1, wherein the inner tube is formed of one of an expanded polyethylene (ePE) and an Aliphatic Thermoplastic Polyether Polyurethane (ATPU).

9. A method of utilizing an introducer sheath assembly comprising an outer tube, an inner tube extending along a longitudinal axis between a first end and a second end, the inner tube comprising a center portion intermediate the first end and the second end and defining a center portion outer diameter, the inner tube is formed of a compliant material and defines a first end ring adjacent the first end, the first end ring defined by longitudinally compressing and fixing a first segment of the inner tube and the first end ring defining a first end ring outer diameter, a pressurizable space defined between the outer tube and the inner tube, a first collar coupled to each of the outer tube and the inner tube such that the inner tube is engaged by the first collar at the first end ring, and a fill port fluidly coupled to the pressurizable space, the method comprising:
providing a tool insertable into the inner tube;
pressurizing the pressurizable space with a fluid input to the fill port; and
reducing the center portion outer diameter in response to the pressurizing such that the center portion sealingly collapses around the tool.

10. The method of claim 9, wherein the outer tube is defined by a flexible material, the method further comprising:
distending the outer tube in response to the pressurizing.

11. The method of claim 9, wherein the inner tube further defines a second end ring adjacent the second end, the second end ring defined by longitudinally compressing and fixing a second segment of the inner tube and the second end ring defining a second end ring outer diameter.

12. The method of claim 11, further comprising a second collar coupled to each of the outer tube and the inner tube such that the inner tube is engaged by the second collar at the second end ring.

13. The method of claim 11, wherein each of the first segment and the second segment are heat treated to fix the first end ring and the second end ring.

14. The method of claim 9, further comprising:
depressurizing the pressurizable space by removing fluid from the pressurizable space through the fill port; and
distending the inner tube in response to the depressurizing.

15. The method of claim 9, wherein the inner tube is formed of one of an expanded polyethylene (ePE) and an Aliphatic Thermoplastic Polyether Polyurethane (ATPU).

16. A method of manufacturing a medical device comprising:
treating an inner tube defining a first end portion, a second end portion, and a center portion such that at least the first end portion is in a reflowable state, wherein the inner tube is drapeable when in a solid state;
manipulating the first end portion while in the reflowable state to form a first end ring;
arranging the inner tube in an outer tube; and
coupling a first collar to the outer tube and the inner tube adjacent the first end ring such that the inner tube and the outer tube define a pressurizable space therebetween.

17. The method of claim 16, further comprising:
treating the inner tube such that the second end portion is in a reflowable state; and
manipulating the second end portion to define a second end ring.

18. The method of claim 16, wherein the first end ring defines a first end ring outer diameter, the center portion defines a center portion outer diameter, and the first end ring outer diameter is greater than the center portion outer diameter.

19. The method of claim 16, wherein the first end ring is formed by longitudinally compressing the first end portion.

20. The method of claim 16, wherein the first end ring is formed by everting a portion of the first end portion.

21. The method of claim 16, wherein the inner tube is formed of one of an expanded polyethylene (ePE) and an Aliphatic Thermoplastic Polyether Polyurethane (ATPU).
